(19) United States
(12) Patent Application Publication
(10) Pub. No.: US 2003/0233008 A1
(43) Pub. Date: Dec. 18, 2003

(54) Process for the preparation of carboxylic benzyl esters

(76) Inventors: Pieter Ooms, Krefeld (DE); Ursula Jansen, Neuss (DE); Bernd-Ulrich Schenke, Bottrop (DE)

Correspondence Address: BAYER CHEMICALS CORPORATION, 100 BAYER ROAD, PITTSBURGH, PA, 15205, US

(21) Appl. No.: 10/458,009

(22) Filed: Jun. 10, 2003

(30) Foreign Application Priority Data
Jun. 12, 2002 (DE) 10226040.0

(51) Int. Cl. C07C 255/49; C07C 69/76; C07C 67/10

(52) U.S. Cl. 558/416; 560/103; 560/236

(57) ABSTRACT

Carboxylic benzyl esters can be prepared by reacting benzyl chloride with carboxylic acids in the presence of one or more quaternary ammonium carboxylates as catalyst.

### BACKGROUND OF THE INVENTION

1. Field of the Invention

The present invention relates to a process for the preparation of carboxylic benzyl esters such as benzyl acetate by reacting benzyl chloride with carboxylic acids in the molar ratio 1:1 to 1:50 at 10 to 200° C. and pressures in the range from 0.1 to 50 bar in the presence of one or more quaternary ammonium carboxylate as a catalyst.

2. Brief Description of the Prior Art

Benzyl acetate, the main component of jasmine oil, is an important odorant for the preparation of fragrance compositions and starting material for the preparation of fruit ethers.

The preparation of benzyl acetate by esterification of benzyl alcohol with acetic acid has been known for a long time.

Benzyl acetate can also be prepared by reacting benzyl chloride with alkali metal acetates (Ullman, Lexikon Chemie, 10^{th} Edition, Volume 2, and page 1217). A disadvantage here is the formation of salts, which have to be disposed of, and thus reduce the profitability of this process.

EP A 0463922 describes the preparation of benzyl acetate from benzyl chloride and acetic acid in the presence of tertiary amines. A disadvantage here is that equimolar amounts of amine are used.

The object here is to develop a process for the preparation of carboxylic benzyl esters starting from benzyl chloride which can be carried out under mild reaction conditions, and which leads to good yields of carboxylic benzyl esters in a cost-effective manner.

### SUMMARY OF THE INVENTION

In accordance with the foregoing, the present invention encompasses a process for the preparation of carboxylic benzyl esters of the formula

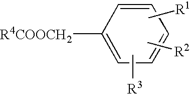

in which

R¹ to R³ are identical or different and are hydrogen, C₁-C₆-alkyl, C₁-C₆-alkoxy, C₁-C₆-haloalkyl, C₁-C₆-haloalkoxy, CN, CO(C₁-C₆-alkyl), NO₂ or halogen and

R⁴ is hydrogen, C₁-C₂₀-alkyl, C₂-C₂₀-alkenyl, C₆-C₁₂-aryl, C₁-C₆-haloalkyl, C₂-C₆-haloalkenyl or C₆-C₁₂-haloaryl,

which carboxylic benzyl esters are from benzyl chloride which is characterized in that benzyl chlorides of the formula

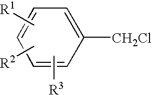

in which

R¹, R² and R³ have the meanings given above, or mixtures of benzyl chlorides and benzyl alcohols of the formula

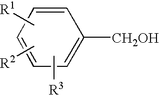

in which

R¹, R² and R³ have the meanings given above are reacted with carboxylic acids of the formula

R⁴COOH

in which

R⁴ has the meaning given above, in the presence of one or more quaternary ammonium carboxylate of the formula

R⁴COONR⁵R⁶R⁷R⁸

in which

R⁵ to R⁸ are identical or different and are C₁-C₆-alkyl, C₃-C₆-cycloalkyl, C₇-C₁₀-aralkyl, C₆-C₁₂-aryl, C₁-C₆-haloalkyl, C₇-C₁₀-haloaralkyl or C₆-C₁₂-haloaryl.

The process according to the invention can be carried out in a cost-effective manner and under mild reaction conditions.

### DETAILED DESCRIPTION OF THE INVENTION

The radicals R¹ to R³ generally have the following meanings:

The alkyl radical is generally a straight-chain or branched hydrocarbon radical having 1 to 6, preferably 1 to 4, especially preferably 1 or 2, carbon atoms. Examples which may be mentioned are methyl, ethyl, propyl, isopropyl, butyl, isobutyl, pentyl, isopentyl, hexyl and isohexyl. Preference is given to methyl, ethyl, propyl, isopropyl, butyl, pentyl and hexyl, and particular preference is given to methyl and ethyl.

Alkoxy radical is generally a straight-chain or branched alkoxy radical having 1 to 6, preferably 1 to 4, especially preferably 1 or 2, carbon atoms. Examples which may be mentioned are methoxy, ethoxy, propoxy, isopropoxy, butoxy, isobutoxy, pentoxy, isopentoxy, hexoxy and isohexoxy. Preference is given to methoxy, ethoxy, propoxy, isopropoxy, butoxy, isobutoxy, pentoxy and hexoxy, and particular preference is given to methoxy and ethoxy.

Haloalkyl radical is generally a straight-chain or branched hydrocarbon radical having 1 to 6, preferably 1 to 4, especially preferably 1 or 2, carbon atoms having 1 to 10, preferably 1 to 8, especially preferably having 1 to 5, halogen atoms. Examples, which may be mentioned, are fluoromethyl, difluoromethyl, trifluoromethyl, fluoroethyl, fluoropropyl and hexafluorobutyl. Preference is given to fluoromethyl, difluoromethyl, trifluoromethyl, fluoroethyl, fluoropropyl and hexafluorobutyl, and particular preference is given to fluoromethyl and trifluoromethyl.

Haloalkoxy is generally a straight-chain or branched alkoxy radical having 1 to 6, preferably 1 to 4, especially preferably 1 or 2, carbon atoms having 1 to 10, preferably 1 to 8, especially preferably having 1 to 5, halogen atoms. Examples which may be mentioned are chloromethoxy, fluoromethoxy, difluoromethoxy, trifluoromethoxy, fluoroethoxy, fluoropropoxy and hexafluorobutoxy. Preference is given to chloromethoxy, fluoromethoxy, trifluoromethoxy, fluoroethoxy, fluoropropoxy and hexafluorobutoxy, and particular preference is given to fluoromethoxy and trifluoromethoxy.

Halogen generally means fluorine, chlorine, bromine and iodine, preferably fluorine, chlorine and bromine, especially fluorine and chlorine.

Very particularly preferred substituents for R¹ to R³are hydrogen, methyl, trifluoromethyl, methoxy, fluorine or chlorine. Very particularly preferred substituents for R⁴ are hydrogen, methyl, ethyl, propyl, butyl, ethenyl, propenyl, benzyl, phenyl, salicyl or styryl.

In accordance with the process of the invention it is possible, for example, to prepare the following carboxylic benzyl esters:

benzyl formate, benzyl acetate, benzyl propionate, benzyl butyrate, benzyl pentanoate, benzyl hexanoate, benzyl heptanoate, benzyl octanoate, benzyl nonanoate, benzyl decanoate, benzyl undecanoate, benzyl dodecanoate, benzyl tridecanoate, benzyl tetradecanoate, benzyl pentadecanoate, benzyl hexadecanoate, benzyl heptadecanoate, benzyl octadecanoate, benzyl nonadecanoate, benzyl 2-hydroxybenzoate, benzyl 3-hydroxybenzoate, benzyl 4-hydroxybenzoate, benzyl 3-chloro-2-hydroxybenzoate, benzyl 4-chloro-2-hydroxybenzoate, benzyl 5-chloro-2-hydroxybenzoate, benzyl 6-chloro-2-hydroxybenzoate, benzyl 3-bromo-2-hydroxybenzoate, benzyl 3-fluoro-2-hydroxybenzoate, benzyl 4-fluoro-2-hydroxybenzoate, benzyl 2-fluoro-3-hydroxybenzoate, benzyl 2-fluoro-4-hydroxybenzoate, benzyl 3-fluoro-2-hydroxybenzoate, benzyl 2-fluoro-5-hydroxybenzoate, benzyl 2-fluoro-6-hydroxybenzoate, benzyl 2-hydroxy-3-methylbenzoate, benzyl 2-hydroxy-4-methylbenzoate, benzyl 3-hydroxy-2-methylbenzoate, benzyl 4-hydroxy-2-methylbenzoate, benzyl 2-fluoro-6-hydroxy-4-methoxybenzoate, benzyl 3-trifluoromethyl-2-hydroxybenzoate, benzyl 4-trifluoromethyl-2-hydroxybenzoate, benzyl 2-trifluoromethyl-3-hydroxy-benzoate, benzyl 2-fluoroethyl-4-hydroxybenzoate and benzyl 4-fluorobutyl-2-hydroxybenzoate.

Benzyl chlorides for the process according to the invention may be unsubstituted or substituted. Examples of the substituted benzyl chloride is one that carries one or more substituents selected from the group consisting of C₁-C₆-alkyl, C₁-C₆-alkoxy, CN, CO(C₁-C₆-alkyl), NO₂ or halogen. Particular preference is given to using unsubstituted benzyl chloride.

In the process according to the invention, it is also possible to use benzyl chloride or benzyl chloride/benzyl alcohol mixtures, as are produced, for example, in the preparation of benzyl alcohol from benzyl chloride. The content of benzyl chloride may be 50 to 100%, preferably 60 to 99%, particularly preferably 70 to 98%.

The carboxylic acids used in the process according to the invention are straight-chain and branched, saturated and unsaturated alkyl-, aralkyl- and arylcarboxylic acids having 1 to 20 carbon atoms, such as, for example, formic acid, acetic acid, propionic acid, butyric acid, isobutyric acid, valeric acid, isovaleric acid, caproic acid, heptanoic acid, caprylic acid, nonanoic acid, capric acid, undecanoic acid, lauric acid, tridecanoic acid, myristic acid, palmitic acid, stearic acid, oleic acid, linoleic acid, chloroacetic acid, linolenic acid, acrylic acid, methacrylic acid, cinnamic acid, phenylacetic acid, benzoic acid or salicylic acid. Preference is given to carboxylic acids having 1 to 15 carbon atoms, particularly preferably 1 to 10 carbon atoms. Very particularly preferred carboxylic acids are formic acid, acetic acid, chloroacetic acid, propionic acid, hexanoic acid and benzoic acid.

The process according to the invention is preferably carried out with removal of the hydrogen chloride that is formed. The hydrogen chloride is preferably removed by passing an inert gas through, such as, for example, nitrogen.

Suitable catalysts for the process according to the invention are quaternary ammonium carboxylates, such as, for example, tetramethylammonium formate, tetramethylammonium propionate, tetramethylammonium butyrate, tetramethyl-ammonium benzoate, tetramethylammonium salicylate, tetramethylammonium acetate, tetraethylammonium acetate, tetrapropylammonium acetate, tetrabutyl-ammonium acetate, benzyltrimethylammonium acetate, dibenzyldimethyl-ammonium acetate, benzyltriethylammonium acetate, benzyltripropylammonium acetate, benzyltributylammonium acetate and benzyltridodecylammonium acetate, where appropriate applied to one or more, and preferably one, support.

Preference is given to tetramethylammonium acetate, tetraethylammonium acetate, tetrapropylammonium acetate, benzyltrimethylammonium acetate and benzyltributylammonium acetate, and particular preference is given to tetra-methylammonium acetate and tetraethylammonium acetate. Methods for the preparation of the quaternary ammonium carboxylates are known and are described, for example, in Japanese patent application JP 62.174.036 and U.S. Pat. No. 5,001,156.

The quaternary ammonium carboxylates can be applied to a support, optionally calcined, or used as the heterogeneous catalyst.

The catalysts can, for example, be used as powders or shaped bodies and are separated off after the reaction by, for example, filtration, sedimentation or centrifugation.

In the case of a fixed-bed arrangement, the quaternary ammonium salts are preferably applied to a support and used as molded bodies, e.g. as spheres, cylinders, rods, hollow cylinders, rings, etc.

The quaternary ammonium salts are used in amounts of 0.1 to 10 equivalents of quaternary ammonium carboxylate, based on benzyl chloride. Preferably, the process according to the invention is carried out with intensive thorough mixing of the reactants. An intensive thorough mixing can be achieved in various ways known to the person skilled in the art, for example by stirring, jets, baffles, static mixers, pumps, turbulent flows in narrow tubes and by ultrasound.

Such devices are described in more detail in Ullmann's Encyclopedia of Industrial Chemistry, 5^{th} Edition, Volume B, Unit Operations, Sections 25, 26, B4 pp. 569 to 572, Verlag Chemie, Weinheim 1988.

In a preferred embodiment of the process according to the invention, quaternary ammonium carboxylate is added to a mixture or suspension of the benzyl chloride and carboxylic acid and, when the reaction is complete, the catalyst is separated off by, for example, filtration or centrifugation or following isolation of the carboxylic benzyl ester.

In a further preferred embodiment of the process according to the invention, it is carried out in the trickle phase and the supported quaternary ammonium carboxylate is present in the form of a fixed-bed catalyst. The catalyst bed is preferably located in a perpendicular tubular reactor, which preferably contains intermediate plates to improve distribution of the liquid stream and to improve wetting of the catalyst bed.

Both in the case of the suspended catalyst and also in fixed-bed process variants, the work-up can be carried out by adding a water-immiscible solvent, preferably toluene, to the reaction products. After the organic phase, which contains the crude carboxylic benzyl ester, has been separated off, it can be further purified, for example, by distillation.

The process according to the invention can be carried out discontinuously, continuously or semi continuously.

The temperature at which the process according to the invention is carried out is preferably 15 to 200° C., particularly preferably 25 to 190° C., very particularly preferably 30 to 180° C.

When the reaction is carried out above 115° C., it is necessary to work under increased pressure corresponding to the vapor pressure. The required superatmospheric pressure is then at least equal to the vapor pressure of the reaction mixture. It may be up to 200 bar, preferably up to 50 bar.

Where appropriate, the process according to the invention can be carried out under a customary protective gas, such as, for example, nitrogen, helium or argon.

The process according to the invention can be explained by the following reaction equation:

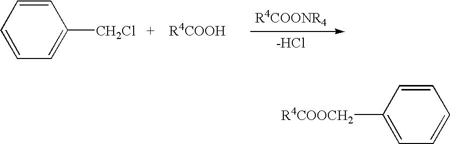

The process according to the invention gives carboxylic benzyl esters in good yields with high conversion and good selectivity. The process according to the invention can be carried out easily without high expenditure on apparatus.

The invention is further described using the following illustrative but non-limiting examples.

### EXAMPLES

The percentages given in the examples below refer to the weight.

### Example 1

63.3 g (0.5 mol) of benzyl chloride, 60.0 g (1.0 mol) of acetic acid and 6.3 g (0.0474 mol) of tetramethylammonium acetate were heated at 120° C. in a flask fitted with baffles and paddle stirrer under vigorous stirring (250 rpm) and under nitrogen. After a reaction time of 3 hours, the mixture was cooled rapidly and the reaction mixture was analysed using gas chromatography. The reaction mixture comprised benzyl acetate and benzyl chloride in the ratio 14:85.

### Example 2

Example 1 was repeated, but with 120.0 g (2.0 mol) of acetic acid. The reaction time was 34 hours. The reaction mixture comprised benzyl acetate and benzyl chloride in the ratio 34:64.

### Example 3

Example 1 was repeated, but with 600.0 g (10.0 mol) of acetic acid. The reaction time was 68 hours. The reaction mixture comprised benzyl acetate and benzyl chloride in the ratio 70:30.

### Example 4

Example 1 was repeated, but with 6.3 g (0.0241 mol) of tetraethylammonium acetate tetrahydrate. The reaction time was 7 hours. The reaction mixture comprised benzyl acetate and benzyl chloride in the ratio 15:84.

### Example 5

Example 1 was repeated, but with 5.5 g (0.0271 mol) of tetrapropylammonium acetate. The reaction time was 7 hours. The reaction mixture comprised benzyl acetate and benzyl chloride in the ratio 15:83.

### Example 6

Example 1 was repeated, but with 6.3 g (0.0209 mol) of tetrabutylammonium acetate. The reaction time was 7 hours. The reaction mixture comprised benzyl acetate and benzyl chloride in the ratio 11:88.

### Example 7

Example 1 was repeated, but with 5.1 g (0.0301 mol) of benzyltrimethylammonium acetate, prepared from 12.7 g of an aqueous 40% strength benzyltrimethylammonium hydroxide solution, 47.0 g (0.46 mol) of acetic anhydride and 6.0 g (0.1 mol) of acetic acid. The reaction time was 7 hours. The reaction mixture comprised benzyl acetate and benzyl chloride in the ratio 14:84.

### Example 8 (Comparison)

Use of a tetraalkylammonium chloride according to J. C. S., Chem. Commun., 1991, p.853, 854; EP-A 0 534 817.

Example 1 was repeated, but with 5.2 g (0.0474 mol) of tetramethylammonium chloride. The reaction time was 3 hours. The reaction mixture comprised benzyl acetate and benzyl chloride in the ratio 6:94.

### Example 9 (Comparison)

Use of a tetraalkylammonium bromide according to J. C. S., Chem. Commun., 1991, p. 853, 854; EP-A 0 534 817.

Example 1 was repeated but with 7.5 g (0.0474 mol) of tetramethylammonium bromide. The reaction time was 3 hours. The reaction mixture comprised benzyl acetate and benzyl chloride in the ratio 10:82.

### Example 10 (Comparison)

Use of a benzyldialkylamine according to EP-A 0 463 922

Example 1 was repeated, but with 6.3 g (0.0470 mol) of benzyldimethylamine. The reaction time was 3 hours. The reaction mixture comprised benzyl acetate and benzyl chloride in the ratio 9:81.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for the preparation of carboxylic benzyl esters of the formula 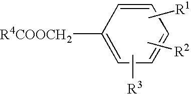
in which
R¹ to R³ are identical or different and are C₁-C₆-alkyl, C₁-C₆-alkoxy, C₁-C₆-haloalkyl, C₁-C₆-haloalkoxy, CN, CO(C₁-C₆-alkyl), NO₂ or halogen and
R⁴ is hydrogen, C₁-C₂₀-alkyl, C₂-C₂₀-alkenyl, C₆-C₁₂-aryl, C₁-C₆-haloalkyl, C₂-C₆-haloalkenyl or C₆-C₁₂-haloaryl,
from benzyl chlorides, comprising reacting benzyl chlorides of the formula 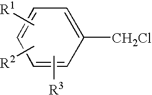
in which
R¹, R² and R³ have the meanings given above, or mixtures of benzyl chlorides and benzyl alcohols of the formula 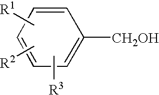
in which
R¹, R² and R³ have the meanings given above with carboxylic acids of the formula
R⁴COOH
in which
R⁴ has the meaning given above,
in the presence of one or more quaternary ammonium carboxylate of the formula
R⁴COONR⁵R⁶R⁷R⁸
in which
R⁵ to R⁸ are identical or different and are C₁-C₆-alkyl, C₃-C₆-cycloalkyl, C₇-C₁₀ aralkyl, C₆-C₁₂-aryl, C₁-C₆-haloalkyl, C₇-C₁₀-haloaralkyl or C₆-C₁₂-haloaryl.

2. Process according to claim 1, characterized in that the quaternary ammonium carboxylate used is tetramethylammonium acetate, tetraethyl-ammonium acetate, tetrapropylammonium acetate, tetrabutylammonium acetate or benzyltrimethylammonium acetate.

3. Process according to claim 1, characterized in that the carboxylic acids used are straight-chain and branched, saturated and unsaturated alkyl-, aralkyl- and arylcarboxylic acids having 1 to 20 carbon atoms, selected from the group consisting of formic acid, acetic acid, propionic acid, butyric acid, isobutyric acid, valeric acid, isovaleric acid, caproic acid, hectanoic acid, caprylic acid, nonanoic acid, capric acid, undecanoic acid, lauric acid, tridecanoic acid, myristic acid, palmitic acid, stearic acid, oleic acid, linoleic acid, chloroacetic acid, linolenic acid, acrylic acid, methacrylic acid, cinnamic acid, phenylacetic acid, benzoic acid and salicylic acid.

4. Process according to claim 1, characterized in that the benzyl chloride is a substituted or unsubstituted benzyl chloride.

5. Process according to claim 1, characterized in that the benzyl chloride is a substituted benzyl chloride which carries one or more substituents selected from the group consisting of C₁-C₆-alkyl, C₁-C₆-alkoxy, CN, CO(C₁-C₆-alkyl), NO₂ or halogen.

6. Process according to claim 1, characterized in that 0.1 to 50 equivalents of carboxylic acid, based on benzyl chloride, are used.

7. Process according to claim 1, characterized in that 0.1 to 10 equivalents of quaternary ammonium carboxylate, based on benzyl chloride, are used.

8. Process according to claim 1, characterized in that the reaction takes place with removal of hydrogen chloride by passing nitrogen through.

9. Process according to claim 1, characterized in that the reaction is carried out at a temperature of from 15 to 200° C.

10. Process according to claim 1, characterized in that the reaction is carried out at a pressure of from 1 to 200 bar.
